(19) United States
(12) Patent Application Publication
(10) Pub. No.: US 2005/0055060 A1
(43) Pub. Date: Mar. 10, 2005

(54) Determination of respiratory characteristics from AV conduction intervals

(76) Inventors: Steve Koh, Rowland Heights, CA (US); Euljoon Park, Stevenson Ranch, CA (US); Gene A. Bornzin, Simi Valley, CA (US)

Correspondence Address: PACESETTER, INC., 15900 VALLEY VIEW COURT, SYLMAR, CA, 91392-9221, US

(21) Appl. No.: 10/656,540

(22) Filed: Sep. 5, 2003

(51) Int. Cl. A61N 1/365

(52) U.S. Cl. 607/17

(57) ABSTRACT

Exemplary methods and devices for determining respiratory and/or autonomic characteristics based at least in part on atrioventricular conduction and/or one or more atrioventricular conduction interval times. Other methods, devices and/or systems are also disclosed.

### TECHNICAL FIELD

Exemplary methods and/or devices presented herein generally relate to determining respiratory and/or autonomic characteristics based at least in part on one or more atrioventricular conduction interval times.

### BACKGROUND

A holistic approach to cardiac health can benefit greatly from knowledge of patient respiration and/or autonomic tone. In particular, approaches that rely on implantable cardiac therapy devices can use such knowledge to deliver improved therapy, which may, in turn, extend patient life. For example, central sleep apnea is known to involve both respiratory and autonomic dysfunction that can severely strain cardiac performance. Knowledge of respiration and/or autonomic state may allow an implantable cardiac device to quickly offset the detrimental effects of central sleep apnea.

To date, most implantable cardiac devices cannot measure or otherwise determine respiratory and/or autonomic characteristics. And, in the few devices capable of such measurements, additional hardware features are typically used to measure respiratory and/or autonomic characteristics directly. Overall, a need exists for methods and/or devices that can readily determine respiratory and/or autonomic characteristics indirectly, for example, through electrocardiogram and/or other cardiac information. Various exemplary methods and/or devices are described below which may address this need and/or other needs.

### SUMMARY

Exemplary methods and devices are disclosed herein for determining respiratory and/or autonomic characteristics based on atrioventricular conduction and/or one or more atrioventricular conduction interval times. Various exemplary devices for performing such exemplary methods are also disclosed herein along with a variety of other exemplary methods and/or devices. In general, the various devices and methods described herein, and equivalents thereof, are suitable for use in a variety of pacing therapies and other cardiac related therapies.

### BRIEF DESCRIPTION OF THE DRAWINGS

Features and advantages of the described implementations can be more readily understood by reference to the following description taken in conjunction with the accompanying drawings.

FIG. 1 is a simplified diagram illustrating an exemplary implantable stimulation device in electrical communication with at least three leads implanted into a patient's heart and at least one other lead for delivering stimulation and/or shock therapy. Of course, an exemplary device with a single lead may be used to implement at least some exemplary methods described herein.

FIG. 2 is a functional block diagram of an exemplary implantable stimulation device illustrating basic elements that are configured to provide cardioversion, defibrillation, pacing stimulation and/or autonomic nerve stimulation or other tissue and/or nerve stimulation. The implantable stimulation device is further configured to sense information and administer stimulation pulses responsive to such information.

FIG. 3 is an approximate anatomical diagram of a heart and a waveform or ECG wherein the waveform includes a P wave and an R wave.

FIG. 4 is an approximate anatomical diagram of a heart and a waveform or ECG wherein the waveform includes an A wave and an R wave.

FIG. 5 is a diagram of an exemplary waveform, including atrial capture and a corresponding A wave to R wave interval (ARI), which may be considered a subset of atrioventricular interval time.

FIG. 6 is a diagram of exemplary waveforms for two different autonomic states for a given atrial pacing rate.

FIG. 7 is a diagram of exemplary waveforms wherein one waveform corresponds to exhalation and another waveform corresponds to inhalation for a given atrial pacing rate.

FIG. 8 is diagram that includes an exemplary plot of AVI times versus time for “normal” respiration and an exemplary plot of AVI times versus time for “abnormal” respiration.

FIG. 9 is an exemplary plot of atrial pacing rate versus respiratory cycle length that includes a curve based on an exemplary sampling theory.

FIG. 10 is an exemplary plot of atrial rate versus respiratory cycle length that includes a curve for intrinsic atrial rate and a curve for atrial paced rate based on an exemplary sampling theory.

FIG. 11 is a flow chart diagram of an exemplary method for determining respiratory characteristics. In an alternative, such a method is suitable for determining autonomic characteristics.

FIG. 12 is a flow chart of another exemplary method for determining respiratory characteristics. In an alternative, such a method is suitable for determining autonomic characteristics.

### DETAILED DESCRIPTION

The following description is of the best mode presently contemplated for practicing the described implementations. This description is not to be taken in a limiting sense, but rather is made merely for the purpose of describing the general principles of the implementations. The scope of the described implementations should be ascertained with reference to the issued claims. In the description that follows, like numerals or reference designators will be used to reference like parts or elements throughout.

Exemplary Stimulation Device

The techniques described below are intended to be implemented in connection with any stimulation device that is configured or configurable to stimulate nerves and/or stimulate and/or shock a patient's heart.

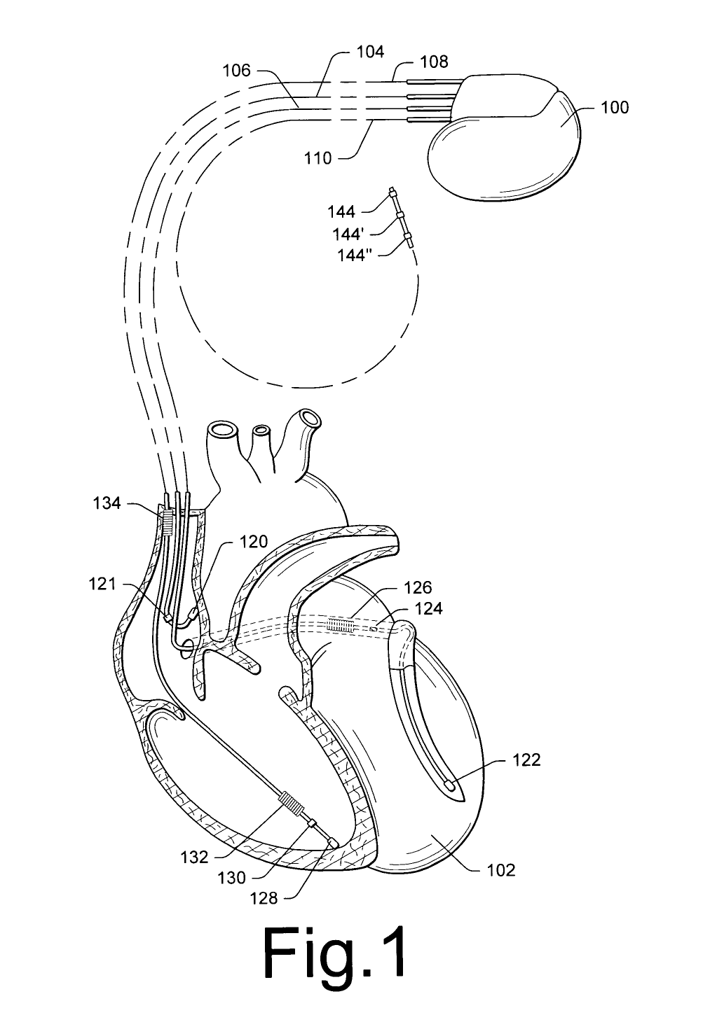

FIG. 1 shows an exemplary stimulation device 100 in electrical communication with a patient's heart 102 by way of three leads 104, 106, 108, suitable for delivering multi-chamber stimulation and shock therapy. The leads 104, 106, 108 are optionally configurable for delivery of stimulation pulses suitable for stimulation of autonomic nerves. In addition, the device 100 includes a fourth lead 110 having, in this implementation, three electrodes 144, 144′, 144″ suitable for stimulation of autonomic nerves and/or detection of other physiologic signals that may be used by the implanted system to modify the pacing parameters. This lead may be positioned in and/or near a patient's heart or near an autonomic nerve within a patient's body and remote from the heart. The right atrial lead 104, as the name implies, is positioned in and/or passes through a patient's right atrium. The right atrial lead 104 optionally senses atrial cardiac signals and/or provide right atrial chamber stimulation therapy. As shown in FIG. 1, the stimulation device 100 is coupled to an implantable right atrial lead 104 having, for example, an atrial tip electrode 120, which typically is implanted in the patient's right atrial appendage. The lead 104, as shown in FIG. 1, also includes an atrial ring electrode 121. Of course, the lead 104 may have other electrodes as well. For example, the right atrial lead optionally includes a distal bifurcation having electrodes suitable for stimulation of autonomic nerves.

To sense atrial cardiac signals, ventricular cardiac signals and/or to provide chamber pacing therapy, particularly on the left side of a patient's heart, the stimulation device 100 is coupled to a coronary sinus lead 106 designed for placement in the coronary sinus and/or tributary veins of the coronary sinus. Thus, the coronary sinus lead 106 is optionally suitable for positioning at least one distal electrode adjacent to the left ventricle and/or additional electrode(s) adjacent to the left atrium. In a normal heart, tributary veins of the coronary sinus include, but may not be limited to, the great cardiac vein, the left marginal vein, the left posterior ventricular vein, the middle cardiac vein, and the small cardiac vein.

Accordingly, an exemplary coronary sinus lead 106 is optionally designed to receive atrial and ventricular cardiac signals and to deliver left ventricular pacing therapy using, for example, at least a left ventricular tip electrode 122, left atrial pacing therapy using at least a left atrial ring electrode 124, and shocking therapy using at least a left atrial coil electrode 126. For a complete description of a coronary sinus lead, the reader is directed to U.S. patent application Ser. No. 09/457,277, filed Dec. 8, 1999, entitled “A Self-Anchoring, Steerable Coronary Sinus Lead” (Pianca et al.); and U.S. Pat. No. 5,466,254, “Coronary Sinus Lead with Atrial Sensing Capability” (Helland), which are incorporated herein by reference. The coronary sinus lead 106 further optionally includes electrodes for stimulation of autonomic nerves. Such a lead may include pacing and autonomic nerve stimulation functionality and may further include bifurcations or legs. For example, an exemplary coronary sinus lead includes pacing electrodes capable of delivering pacing pulses to a patient's left ventricle and at least one electrode capable of stimulating an autonomic nerve. An exemplary coronary sinus lead (or left ventricular lead or left atrial lead) may also include at least one electrode capable of stimulating an autonomic nerve; such an electrode may be positioned on the lead or a bifurcation or leg of the lead.

Stimulation device 100 is also shown in electrical communication with the patient's heart 102 by way of an implantable right ventricular lead 108 having, in this exemplary implementation, a right ventricular tip electrode 128, a right ventricular ring electrode 130, a right ventricular (RV) coil electrode 132, and an SVC coil electrode 134. Typically, the right ventricular lead 108 is transvenously inserted into the heart 102 to place the right ventricular tip electrode 128 in the right ventricular apex so that the RV coil electrode 132 will be positioned in the right ventricle and the SVC coil electrode 134 will be positioned in the superior vena cava. Accordingly, the right ventricular lead 108 is capable of sensing or receiving cardiac signals, and delivering stimulation in the form of pacing and shock therapy to the right ventricle. An exemplary right ventricular lead may also include at least one electrode capable of stimulating an autonomic nerve; such an electrode may be positioned on the lead or a bifurcation or leg of the lead.

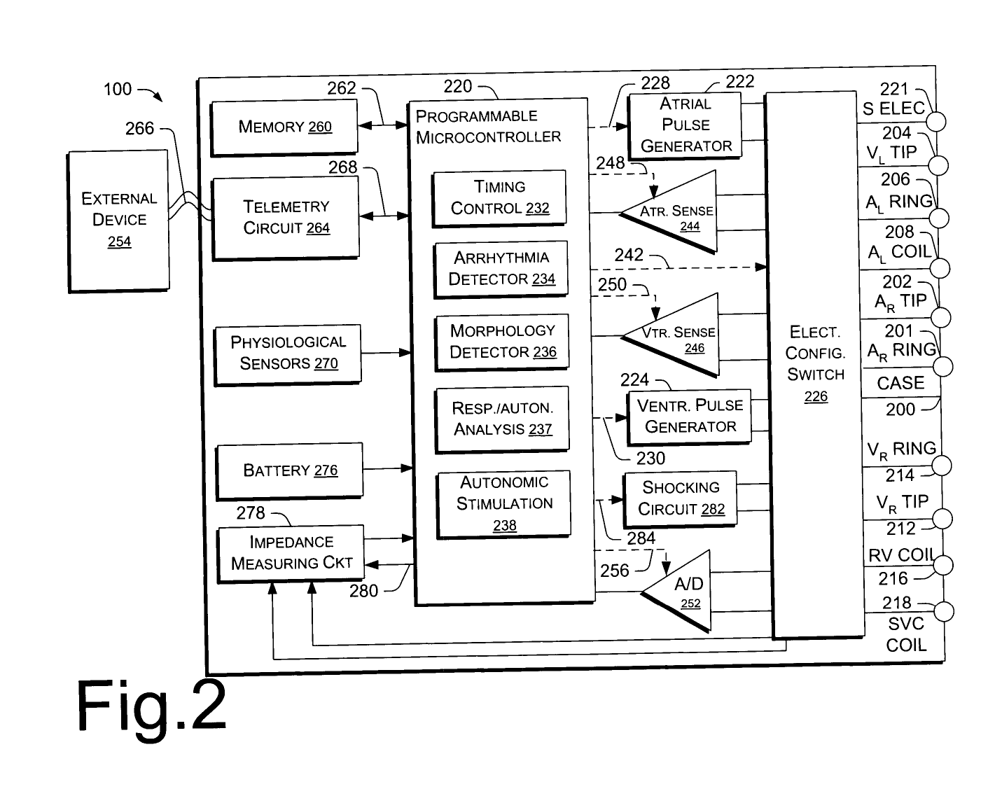

FIG. 2 shows an exemplary, simplified block diagram depicting various components of stimulation device 100. The stimulation device 100 can be capable of treating both fast and slow arrhythmias with stimulation therapy, including cardioversion, defibrillation, and pacing stimulation. The stimulation device can be solely or further capable of delivering stimuli to autonomic nerves. While a particular multi-chamber device is shown, it is to be appreciated and understood that this is done for illustration purposes only. Thus, the techniques and methods described below can be implemented in connection with any suitably configured or configurable stimulation device. Accordingly, one of skill in the art could readily duplicate, eliminate, or disable the appropriate circuitry in any desired combination to provide a device capable of treating the appropriate chamber(s) or regions of a patient's heart with cardioversion, defibrillation, pacing stimulation, and/or autonomic nerve stimulation.

Housing 200 for stimulation device 100 is often referred to as the “can”, “case” or “case electrode”, and may be programmably selected to act as the return electrode for all “unipolar” modes. Housing 200 may further be used as a return electrode alone or in combination with one or more of the coil electrodes 126, 132 and 134 for shocking purposes. Housing 200 further includes a connector (not shown) having a plurality of terminals 201, 202, 204, 206, 208, 212, 214, 216, 218, 221 (shown schematically and, for convenience, the names of the electrodes to which they are connected are shown next to the terminals).

To achieve right atrial sensing, pacing and/or autonomic stimulation, the connector includes at least a right atrial tip terminal (AR TIP) 202 adapted for connection to the atrial tip electrode 120. A right atrial ring terminal (AR RING) 201 is also shown, which is adapted for connection to the atrial ring electrode 121. To achieve left chamber sensing, pacing, shocking, and/or autonomic stimulation, the connector includes at least a left ventricular tip terminal (VL TIP) 204, a left atrial ring terminal (AL RING) 206, and a left atrial shocking terminal (AL COIL) 208, which are adapted for connection to the left ventricular tip electrode 122, the left atrial ring electrode 124, and the left atrial coil electrode 126, respectively. Connection to suitable autonomic nerve stimulation electrodes is also possible via these and/or other terminals (e.g., via a nerve stimulation terminal S ELEC 221).

To support right chamber sensing, pacing, shocking, and/or autonomic nerve stimulation, the connector further includes a right ventricular tip terminal (VR TIP) 212, a right ventricular ring terminal (VR RING) 214, a right ventricular shocking terminal (RV COIL) 216, and a superior vena cava shocking terminal (SVC COIL) 218, which are adapted for connection to the right ventricular tip electrode 128, right ventricular ring electrode 130, the RV coil electrode 132, and the SVC coil electrode 134, respectively. Connection to suitable autonomic nerve stimulation electrodes is also possible via these and/or other terminals (e.g., via the nerve stimulation terminal S ELEC 221).

At the core of the stimulation device 100 is a programmable microcontroller 220 that controls the various modes of stimulation therapy. As is well known in the art, microcontroller 220 typically includes a microprocessor, or equivalent control circuitry, designed specifically for controlling the delivery of stimulation therapy, and may further include RAM or ROM memory, logic and timing circuitry, state machine circuitry, and I/O circuitry. Typically, microcontroller 220 includes the ability to process or monitor input signals (data or information) as controlled by a program code stored in a designated block of memory. The type of microcontroller is not critical to the described implementations. Rather, any suitable microcontroller 220 may be used that carries out the functions described herein. The use of microprocessor-based control circuits for performing timing and data analysis functions are well known in the art.

Representative types of control circuitry that may be used in connection with the described embodiments can include the microprocessor-based control system of U.S. Pat. No. 4,940,052 (Mann et al.), the state-machine of U.S. Pat. No. 4,712,555 (Thornander et al.) and U.S. Pat. No. 4,944,298 (Sholder), all of which are incorporated by reference herein. For a more detailed description of the various timing intervals used within the stimulation device and their inter-relationship, see U.S. Pat. No. 4,788,980 (Mann et al.), also incorporated herein by reference.

FIG. 2 also shows an atrial pulse generator 222 and a ventricular pulse generator 224 that generate pacing stimulation pulses for delivery by the right atrial lead 104, the coronary sinus lead 106, and/or the right ventricular lead 108 via an electrode configuration switch 226. It is understood that in order to provide stimulation therapy in each of the four chambers of the heart (or to autonomic nerves) the atrial and ventricular pulse generators, 222 and 224, may include dedicated, independent pulse generators, multiplexed pulse generators, or shared pulse generators. The pulse generators 222 and 224 are controlled by the microcontroller 220 via appropriate control signals 228 and 230, respectively, to trigger or inhibit the stimulation pulses.

Microcontroller 220 further includes timing control circuitry 232 to control the timing of the stimulation pulses (e.g., pacing rate, atrio-ventricular (AV) delay, interatrial conduction (A-A) delay, or interventricular conduction (V-V) delay, etc.) as well as to keep track of the timing of refractory periods, blanking intervals, noise detection windows, evoked response windows, alert intervals, marker channel timing, etc., which is well known in the art.

Microcontroller 220 further includes an arrhythmia detector 234, a morphology detector 236, and optionally an orthostatic compensator and a minute ventilation (MV) response module; the latter two are not shown in FIG. 2. These components can be utilized by the stimulation device 100 for determining desirable times to administer various therapies, including those to reduce the effects of orthostatic hypotension. The aforementioned components may be implemented in hardware as part of the microcontroller 220, or as software/firmware instructions programmed into the device and executed on the microcontroller 220 during certain modes of operation.

Microcontroller 220 further includes a respiratory and/or autonomic characteristics analysis module 237. The respiratory and/or autonomic characteristics analysis module 237 optionally implements one or more methods for sensing, information analysis, and/or stimulation control. For example, the respiratory and/or autonomic characteristics analysis module 237 optionally implements one or more of the exemplary methods described below.

Microcontroller 220 further includes an autonomic nerve stimulation module 238 for performing a variety of tasks related to autonomic nerve stimulation. This component can be utilized by the stimulation device 100 for determining desirable times to administer various therapies, including, but not limited to, parasympathetic stimulation. The autonomic module 238 may be implemented in hardware as part of the microcontroller 220, or as software/firmware instructions programmed into the device and executed on the microcontroller 220 during certain modes of operation.

The electronic configuration switch 226 includes a plurality of switches for connecting the desired electrodes to the appropriate I/O circuits, thereby providing complete electrode programmability. Accordingly, switch 226, in response to a control signal 242 from the microcontroller 220, determines the polarity of the stimulation pulses (e.g., unipolar, bipolar, etc.) by selectively closing the appropriate combination of switches (not shown) as is known in the art.

Atrial sensing circuits 244 and ventricular sensing circuits 246 may also be selectively coupled to the right atrial lead 104, coronary sinus lead 106, and the right ventricular lead 108, through the switch 226 for detecting the presence of cardiac activity in each of the four chambers of the heart. Accordingly, the atrial (ATR. SENSE) and ventricular (VTR. SENSE) sensing circuits, 244 and 246, may include dedicated sense amplifiers, multiplexed amplifiers, or shared amplifiers. Switch 226 determines the “sensing polarity” of the cardiac signal by selectively closing the appropriate switches, as is also known in the art. In this way, the clinician may program the sensing polarity independent of the stimulation polarity. The sensing circuits (e.g., 244 and 246) are optionally capable of obtaining information indicative of tissue capture.

Each sensing circuit 244 and 246 preferably employs one or more low power, precision amplifiers with programmable gain and/or automatic gain control, bandpass filtering, and a threshold detection circuit, as known in the art, to selectively sense the cardiac signal of interest. The automatic gain control enables the device 100 to deal effectively with the difficult problem of sensing the low amplitude signal characteristics of atrial or ventricular fibrillation.

The outputs of the atrial and ventricular sensing circuits 244 and 246 are connected to the microcontroller 220, which, in turn, is able to trigger or inhibit the atrial and ventricular pulse generators 222 and 224, respectively, in a demand fashion in response to the absence or presence of cardiac activity in the appropriate chambers of the heart. Furthermore, as described herein, the microcontroller 220 is also capable of analyzing information output from the sensing circuits 244 and 246 and/or the data acquisition system 252 to determine or detect whether and to what degree tissue capture has occurred and to program a pulse, or pulses, in response to such determinations. The sensing circuits 244 and 246, in turn, receive control signals over signal lines 248 and 250 from the microcontroller 220 for purposes of controlling the gain, threshold, polarization charge removal circuitry (not shown), and the timing of any blocking circuitry (not shown) coupled to the inputs of the sensing circuits, 244 and 246, as is known in the art.

For arrhythmia detection, the device 100 utilizes the atrial and ventricular sensing circuits, 244 and 246, to sense cardiac signals to determine whether a rhythm is physiologic or pathologic. In reference to arrhythmias, as used herein, “sensing” is reserved for the noting of an electrical signal or obtaining data (information), and “detection” is the processing (analysis) of these sensed signals and noting the presence of an arrhythmia. The timing intervals between sensed events (e.g., P-waves, R-waves, and depolarization signals associated with fibrillation which are sometimes referred to as “F-waves” or “Fib-waves”) are then classified by the arrhythmia detector 234 of the microcontroller 220 by comparing them to a predefined rate zone limit (i.e., bradycardia, normal, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, anti-tachycardia pacing, cardioversion shocks or defibrillation shocks, collectively referred to as “tiered therapy”). Similar rules can be applied to the atrial channel to determine if there is an atrial tachyarrhythmia or atrial fibrillation with appropriate classification and intervention.

Cardiac signals are also applied to inputs of an analog-to-digital (A/D) data acquisition system 252. The data acquisition system 252 is configured to acquire intracardiac electrogram signals, convert the raw analog data into a digital signal, and store the digital signals for later processing and/or telemetric transmission to an external device 254. The data acquisition system 252 is coupled to the right atrial lead 104, the coronary sinus lead 106, the right ventricular lead 108 and/or the nerve stimulation lead through the switch 226 to sample cardiac signals across any pair of desired electrodes.

The microcontroller 220 is further coupled to a memory 260 by a suitable data/address bus 262, wherein the programmable operating parameters used by the microcontroller 220 are stored and modified, as required, in order to customize the operation of the stimulation device 100 to suit the needs of a particular patient. Such operating parameters define, for example, pacing pulse amplitude, pulse duration, electrode polarity, rate, sensitivity, automatic features, arrhythmia detection criteria, and the amplitude, waveshape, number of pulses, and vector of each shocking pulse to be delivered to the patient's heart 102 within each respective tier of therapy. One feature of the described embodiments is the ability to sense and store a relatively large amount of data (e.g., from the data acquisition system 252), which data may then be used for subsequent analysis to guide the programming of the device.

Advantageously, the operating parameters of the implantable device 100 may be non-invasively programmed into the memory 260 through a telemetry circuit 264 in telemetric communication via communication link 266 with the external device 254, such as a programmer, transtelephonic transceiver, or a diagnostic system analyzer. The microcontroller 220 activates the telemetry circuit 264 with a control signal 268. The telemetry circuit 264 advantageously allows intracardiac electrograms and status information relating to the operation of the device 100 (as contained in the microcontroller 220 or memory 260) to be sent to the external device 254 through an established communication link 266.

The stimulation device 100 can further include a physiologic sensor 270, commonly referred to as a “rate-responsive” sensor because it is typically used to adjust pacing stimulation rate according to the exercise state of the patient. However, the physiological sensor 270 may further be used to detect changes in cardiac output (see, e.g., U.S. Pat. No. 6,314,323, entitled “Heart stimulator determining cardiac output, by measuring the systolic pressure, for controlling the stimulation”, to Ekwall, issued Nov. 6, 2001, which discusses a pressure sensor adapted to sense pressure in a right ventricle and to generate an electrical pressure signal corresponding to the sensed pressure, an integrator supplied with the pressure signal which integrates the pressure signal between a start time and a stop time to produce an integration result that corresponds to cardiac output), changes in the physiological condition of the heart, or diurnal changes in activity (e.g., detecting sleep and wake states). Accordingly, the microcontroller 220 responds by adjusting the various pacing parameters (such as rate, AV Delay, V-V Delay, etc.) at which the atrial and ventricular pulse generators, 222 and 224, generate stimulation pulses.

While shown as being included within the stimulation device 100, it is to be understood that the physiologic sensor 270 may also be external to the stimulation device 100, yet still be implanted within or carried by the patient. Examples of physiologic sensors that may be implemented in device 100 include known sensors that, for example, sense respiration rate, pH of blood, ventricular gradient, cardiac output, preload, afterload, contractility, and so forth. Another sensor that may be used is one that detects activity variance, wherein an activity sensor is monitored diurnally to detect the low variance in the measurement corresponding to the sleep state. For a complete description of the activity variance sensor, the reader is directed to U.S. Pat. No. 5,476,483 (Bornzin et al.), issued Dec. 19, 1995, which patent is hereby incorporated by reference.

More specifically, the physiological sensors 270 optionally include sensors for detecting movement and minute ventilation in the patient. The physiological sensors 270 may include a position sensor and/or a minute ventilation (MV) sensor to sense minute ventilation, which is defined as the total volume of air that moves in and out of a patient's lungs in a minute. Signals generated by the position sensor and MV sensor are passed to the microcontroller 220 for analysis in determining whether to adjust the pacing rate, etc. The microcontroller 220 monitors the signals for indications of the patient's position and activity status, such as whether the patient is climbing upstairs or descending downstairs or whether the patient is sitting up after lying down.

The stimulation device additionally includes a battery 276 that provides operating power to all of the circuits shown in FIG. 2. For the stimulation device 100, which employs shocking therapy, the battery 276 is capable of operating at low current drains for long periods of time (e.g., preferably less than 10 μA), and is capable of providing high-current pulses (for capacitor charging) when the patient requires a shock pulse (e.g., preferably, in excess of 2 A, at voltages above 2 V, for periods of 10 seconds or more). The battery 276 also desirably has a predictable discharge characteristic so that elective replacement time can be detected.

The stimulation device 100 can further include magnet detection circuitry (not shown), coupled to the microcontroller 220, to detect when a magnet is placed over the stimulation device 100. A magnet may be used by a clinician to perform various test functions of the stimulation device 100 and/or to signal the microcontroller 220 that the external programmer 254 is in place to receive or transmit data to the microcontroller 220 through the telemetry circuits 264.

The stimulation device 100 further includes an impedance measuring circuit 278 that is enabled by the microcontroller 220 via a control signal 280. The known uses for an impedance measuring circuit 278 include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; and detecting the opening of heart valves, etc. The impedance measuring circuit 278 is advantageously coupled to the switch 226 so that any desired electrode may be used.

In the case where the stimulation device 100 is intended to operate as an implantable cardioverter/defibrillator (ICD) device, it detects the occurrence of an arrhythmia, and automatically applies an appropriate therapy to the heart aimed at terminating the detected arrhythmia. To this end, the microcontroller 220 further controls a shocking circuit 282 by way of a control signal 284. The shocking circuit 282 generates shocking pulses of low (e.g., up to 0.5 J), moderate (e.g., 0.5 J to 10 J), or high energy (e.g., 11 J to 40 J), as controlled by the microcontroller 220. Such shocking pulses are applied to the patient's heart 102 through at least two shocking electrodes, and as shown in this embodiment, selected from the left atrial coil electrode 126, the RV coil electrode 132, and/or the SVC coil electrode 134. As noted above, the housing 200 may act as an active electrode in combination with the RV electrode 132, or as part of a split electrical vector using the SVC coil electrode 134 or the left atrial coil electrode 126 (i.e., using the RV electrode as a common electrode).

Cardioversion level shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of approximately 5 J to 40 J), delivered asynchronously (since R-waves may be too disorganized), and pertaining exclusively to the treatment of fibrillation. Accordingly, the microcontroller 220 is capable of controlling the synchronous or asynchronous delivery of the shocking pulses.

Heart Rhythm

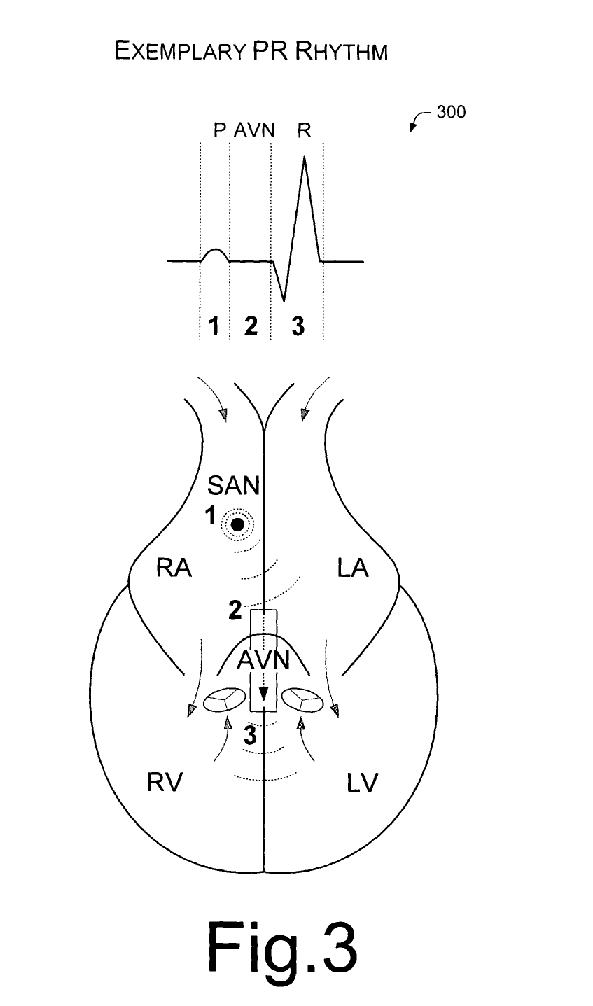

Referring to FIG. 3, an approximate anatomical diagram of a heart and a PR waveform 300 are shown. Action potentials propagating through a normal heart are labeled as follows: 1, associated with the sinoatrial node (SAN) and the atria; 2, associated with the atrioventricular node and/or atrioventricular bundle (AVN); and 3, associated with the ventricles. In a normal heart, cells of the SAN (1) spontaneously depolarize and thereby initiate an action potential (shown as dashed lines emanating from the SAN). This action potential propagates rapidly through the atria (which contract), slowly through the AVN (2) and then to the ventricles (3), which causes ventricular contraction. Thus, in a normal heart, ventricular rhythm relies on conduction of action potentials through the AV node and AV bundle (collectively referred to as the AV node or AVN).

An electrocardiogram (ECG) of normal heart activity (e.g., polarization, depolarization, etc.) typically shows atrial depolarization as a “P wave” and ventricular depolarization as an “R wave”, or QRS complex. The time span between a P wave and an R wave typically depends on AVN conduction and/or heart rate (e.g., rate of SAN).

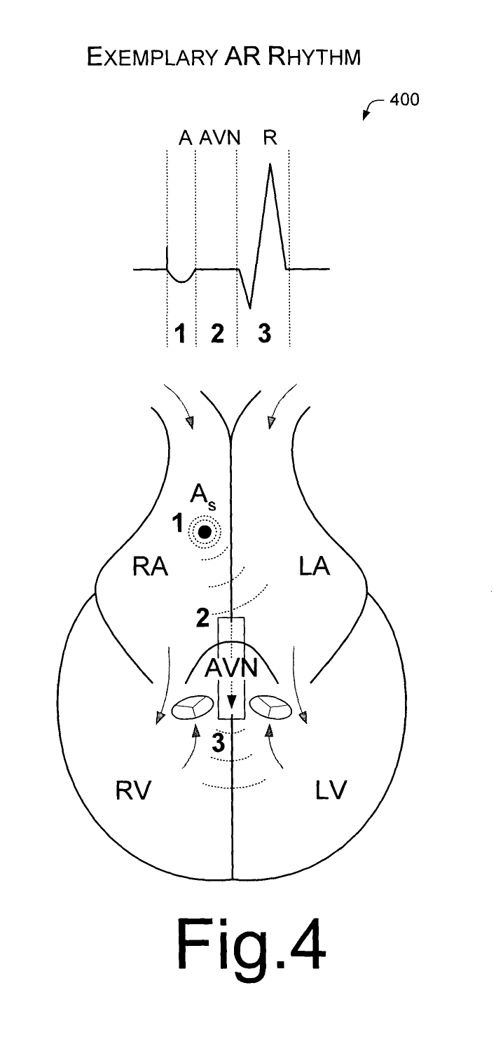

Referring to FIG. 4, an approximate anatomical diagram of a heart, fitted with an atrial pacing device, and an AR waveform 400 are shown. Action potentials propagating through the heart are labeled as follows: 1, associated with a paced atrial stimulus and the atria; 2, associated with the atrioventricular node and/or atrioventricular bundle (AVN); and 3, associated with the ventricles. In an atrial paced heart, cells depolarize near a pacing site (1) and thereby initiate an action potential (shown as dashed lines emanating from the pacing site). This action potential propagates rapidly through the atria (which contract), slowly through the AVN (2) and then to the ventricles (3), which causes ventricular contraction. Thus, in this particular atrial paced heart, ventricular rhythm relies on conduction of action potentials through the AV node and AV bundle (collectively referred to as the AV node or AVN).

An electrocardiogram (ECG) of atrial paced heart activity (e.g., polarization, depolarization, etc.) typically shows atrial depolarization as an “A wave” and ventricular depolarization as an “R wave”, or QRS complex. The time span between an A wave and an R wave typically depends on AVN conduction and/or heart rate (e.g., rate of atrial pacing, A_{S}).

Thus, in the exemplary hearts and waveforms of FIGS. 3 and 4, ventricular function depends on AVN conduction. The AV node is a small subendocardial structure within the interatrial septum, anterior and superior to the coronary sinus, located at the convergence of specialized conduction tracts that course through the atria. The AV node has extensive autonomic innervation and an abundant blood supply from the large AV nodal artery, which is a branch of the right coronary artery in approximately 90 percent of the population, and from septal branches of the left anterior descending coronary artery. The AV node forms part of the only “normal” electrical connection between atria and ventricles. While various conduction sub- or accessory pathways may exist in the AV node or AV nodal region, the AV node is known to transmit impulses slowly via at least one pathway, e.g., requiring approximately 60 ms to approximately 130 ms to traverse about 1 cm of node tissue. In general, slowing of an impulse by AV nodal tissue protects the ventricles by typically not allowing all impulses through, which, in turn, prevents the ventricles from racing in response to a rapid atrial rhythm. The AV nodal tissue also provides time for atrial contraction to occur to facilitate ventricular filling before ventricular contraction begins. Under some circumstances, the AV node blocks all impulses to the ventricles, which may result in a clinical condition requiring permanent pacing for management. Further, radiofrequency ablation of the AV node can also block all impulses to the ventricles. This is an intentional interventional procedure suited to treatment of recurrent atrial tachyarrhythmias where the ventricular response to those arrhythmias cannot be managed through use of other techniques.

En route to the ventricles, action potentials pass via specialized conduction fibers (right bundle branch, left bundle branch, left anterior superior fascicle, left posterior inferior fascicle) ending in Purkinje fibers. The AV node merges in to the Bundle of His (His Bundle) and these fibers then separate into the ventricular specialized conduction system (bundle branches). Blood supplies the AV bundle from the AV nodal artery and septal branches of the left anterior descending artery. The AV bundle has significant autonomic innervations and is somewhat insulated within a collagenous skeleton. Destruction of the AV bundle, for example, through ablation, may also block all impulses to the ventricles.

In general, AV conduction blocks are categorized as first-degree, second-degree and third-degree. First-degree block is associated with P wave to R wave prolongation (e.g., greater than approximately 0.2 s) with all P waves followed by QRST. There are three levels of second-degree block. These are classified as type I (Wenckebach), type II or high-grade. Type II involves intermittent blocking of P waves with constant P-R intervals on those conducted complexes. In addition, for a diagnosis of type 11, there has to be at least two consecutively conducted P waves. In type I (Wenckebach), there is group beating characterized by a stable atrial rate, a progressive lengthening of the PR interval followed by block or non-conduction of a P wave. This results in a relative pause and the cycle starts over again. The first PR complex of the next cycle has the shortest PR interval. In high grade second degree AV bloc, there is either a 2:1 AV block pattern which has constant P-R intervals on the conducted cycles but every second P wave is blocked. High-grade second degree AV block may also be present when two or more successive P waves are blocked and, in general, the atrial rate exceeds the ventricular rate. Third-degree block is associated with complete dissociation of P waves and QRS complexes, and, in general, the atrial rate exceeds the ventricular rate. As described herein, various exemplary methods and/or devices are suitable for use in patients having no AV block and/or first or second degree AV block.

Autonomic Nervous System

As already mentioned, the autonomic nervous system can affect operation of the AV node and/or AV nodal region. The autonomic nervous system includes sympathetic and parasympathetic pathways. Both pathways include afferent pathways (e.g., from an organ to central neurons) and efferent pathways (e.g., postganglionic neurons from ganglia to an organ) that relate to functioning of the heart. For example, parasympathetic efferent postganglionic neurons, when stimulated, suppress atrial rate and contractile force, atrio-ventricular nodal conduction, and ventricular contractile force, also known as contractility or inotropy. Sympathetic efferent postganglionic neurons, when stimulated, generally increase heart rate and increase contractility. Note that contractility is associated with the term “inotropy”, heart rate is associated with the term “chronotropy” and conduction velocity is associated with the term “dromotropy”.

As already mentioned, the stimulation of parasympathetic nerves can act to decrease heart rate while stimulation of sympathetic nerves can act to increase heart rate. In addition, as noted by Mendelowitz, “Advances in parasympathetic control of heart rate and cardiac function”, News Physiol. Sci., 14:155-161 (1999), “when both parasympathetic and sympathetic activity are present, parasympathetic activity generally dominates” and “increases in parasympathetic activity to the heart evoke a bradycardia that is more pronounced when there is a high level of sympathetic firing”. Mendelowitz also noted that “the release of acetylcholine from parasympathetic neurons might act presynaptically to inhibit the release of norepinephrine from sympathetic nerve terminals”.

Regarding sympathetic stimulation, norepinephrine is released by sympathetic nerves. After its release, norepinephrine acts on the sinoatrial node (SA node) to increase the rate of diastolic depolarization, and thereby increase heart rate, and acts on the atrioventricular node (AV node) to increase the velocity of conduction and diminish the refractory period during which the AV node is unresponsive to stimuli coming from the atrium.

Cardiac Intervals

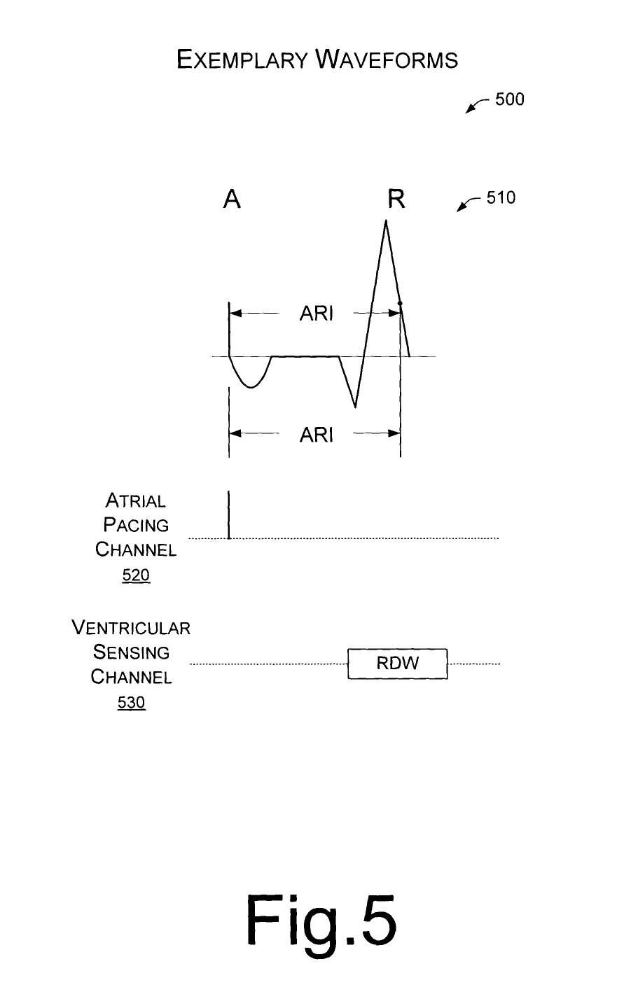

FIG. 5 shows an exemplary waveform 510, an atrial pacing channel 520 and a ventricular sensing channel 530. The exemplary waveform shows an A wave followed by an R wave along with an atrioventricular interval. The atrioventricular interval (AVI) referred to herein is typically defined by an A wave/atrial stimulus to sensed R wave interval (ARI), which is commonly a subset of the AVI. In this example, the atrial pacing channel 520 delivers an atrial pacing stimulus, which is used to begin timing of an ARI. At some time thereafter, the ventricular sensing channel 530 initiates an R wave detection window (RDW). This detection window optionally has a fixed or a programmable duration and optionally varies depending on pacing rate or other factors, for example, AV nodal conduction templates may assist in determining window timing and/or duration. If an R wave is detected within this detection window, then timing of the ARI may end and an appropriate ARI time or AVI time may be assigned for the instant cardiac cycle. Of course, other sensed portions of an atrial or a ventricular waveform (e.g., near-field and/or far-field) may be used to measure or otherwise determine an AVI.

AVI with Respect to Autonomic Activity and Respiration

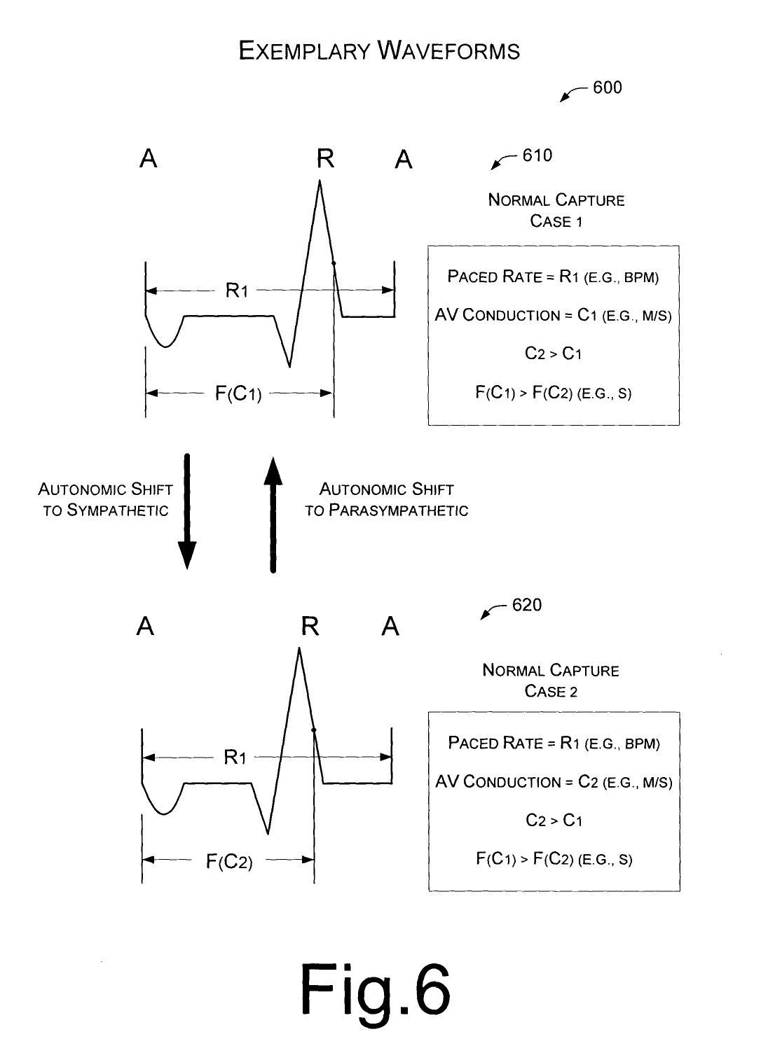

FIG. 6 shows a scenario 600 that includes two approximate waveforms 610, 620 for a first case (Case 1) and a second case (Case 2). Case 1 corresponds to a shift in autonomic tone or autonomic balance towards parasympathetic while Case 2 corresponds to a shift in autonomic tone or autonomic balance towards sympathetic. The Case 1 waveform 610 includes an A wave to A wave interval (AAI) for a paced atrial rate R1 (e.g., in beats per minute) and an A wave to R wave interval (ARI) having a corresponding AV conduction C1 (e.g., in meters per second). In Case 1, the ARI is a function of C1, as represented by the interval time F(C1) (e.g., in seconds). The Case 2 waveform 620 includes an A wave to A wave interval (AAI) for the same paced atrial rate R1 (e.g., in beats per minute) and an A wave to R wave interval (ARI) having a corresponding AV conduction C2 (e.g., in meters per second). In the Case 2 waveform, the ARI is a function of C2, as represented by the interval time F(C2) (e.g., in seconds).

According to the scenario 600, AV conduction is a function of autonomic balance wherein a shift to parasympathetic (Case 1) results in a slowing of AV conduction (i.e., a lesser AV conduction rate) and a shift to sympathetic (Case 2) results in a quickening of AV conduction (i.e., greater AV conduction rate). Consequently, for a set pacing rate, R1, the AV interval (AVI) time lengthens for a shift to parasympathetic (Case 1) and shortens for a shift to sympathetic (Case 2). Thus, during atrial pacing, AV interval (AVI) times can be used to determine whether a patient is experiencing a shift in autonomic balance.

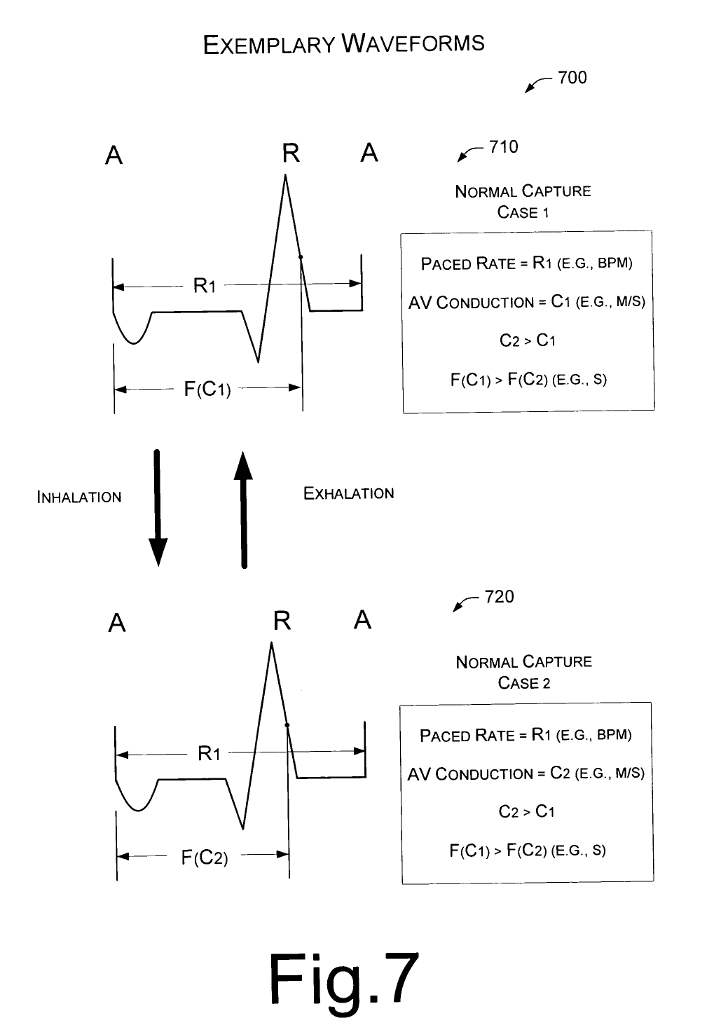

FIG. 7 shows a scenario 700 that includes two approximate waveforms 710, 720 for a first case (Case 1) and a second case (Case 2). Case 1 corresponds to exhalation while Case 2 corresponds to inhalation. The Case 1 waveform 710 includes an A wave to A wave interval (AAI) for a paced atrial rate R1 (e.g., in beats per minute) and an A wave to R wave interval (ARI) having a corresponding AV conduction C1 (e.g., in meters per second). In Case 1, the ARI is a function of C1, as represented by the interval time F(C1) (e.g., in seconds). The Case 2 waveform 720 includes an A wave to A wave interval (AAI) for the same paced atrial rate R1 (e.g., in beats per minute) and an A wave to R wave interval (ARI) having a corresponding AV conduction C2 (e.g., in meters per second). In the Case 2 waveform, the ARI is a function of C2, as represented by the interval time F(C2) (e.g., in seconds).

According to the scenario 700, AV conduction is a function of respiration wherein exhalation (Case 1) results in a slowing of AV conduction (i.e., a lesser AV conduction rate) and inhalation (Case 2) results in a quickening of AV conduction (i.e., greater AV conduction rate). Consequently, for a set pacing rate, R1, the AV interval (AVI) time lengthens for exhalation (Case 1) and shortens for inhalation (Case 2). Thus, during atrial pacing, AV interval (AVI) times can be used to determine whether a patient is exhaling or inhaling.

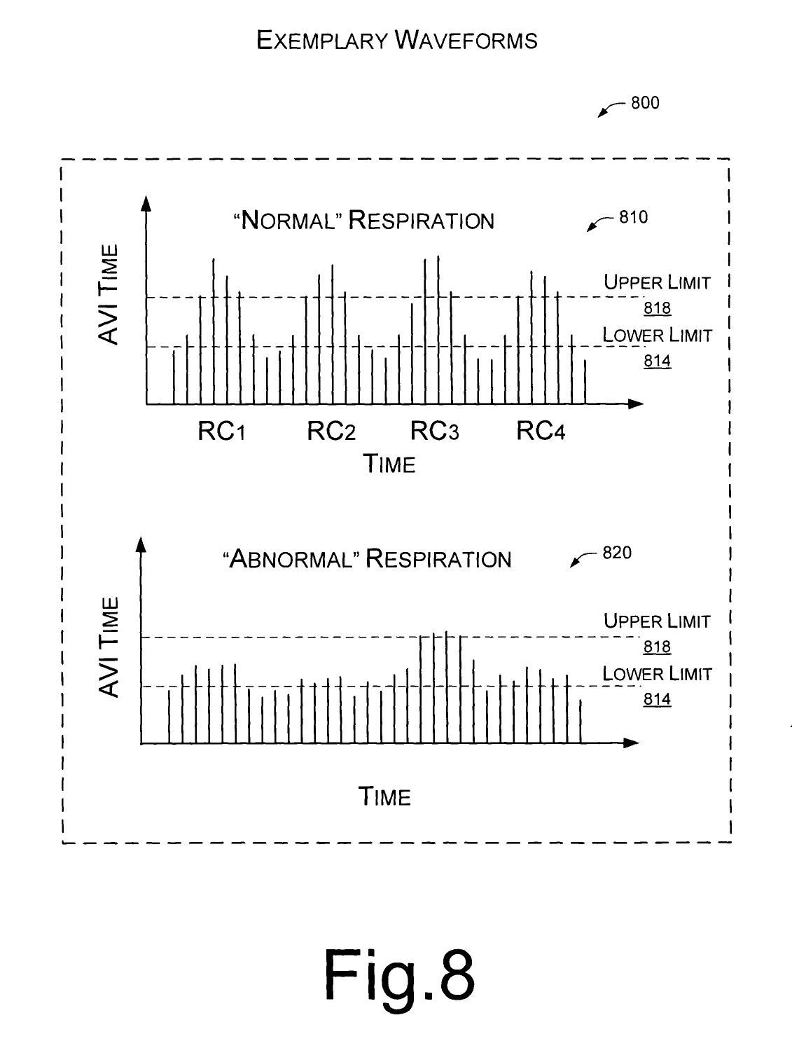

FIG. 8 shows a set of exemplary scenarios 800 that includes a plot of AVI time versus time during “normal” respiration 810 and a plot of AVI time versus time during “abnormal” respiration 820. The normal respiration plot 810 includes indicia of respiratory cycles, labeled RC1, RC2, RC3 and RC4. Each of the respiratory cycles includes about seven AVI times. The plot 810 also shows a lower AVI time limit 814 and an upper AVI time limit 818. In the plot 810, some of the AVI times are greater than the upper AVI time limit 818 and some of the AVI times are less than the lower AVI time limit 814. Such limits are optionally used to determine respiratory cycle characteristics (e.g., cycle length, etc.). Of course, a variety of other techniques may be used to determine respiratory cycle characteristics.

The abnormal respiration plot 820 does not include any regular indicia of respiratory cycles and, instead, includes indicia of abnormal respiration. For example, a patient experiencing central apnea may exhibit AVI times such as those shown in the abnormal respiration plot 820. The plot 820 also shows a lower AVI time limit 914 and an upper AVI time limit 918. In the plot 820, a few of the AVI times are greater than the upper AVI time limit 918 and some of the AVI times are less than the lower AVI time limit 914. Such limits are optionally used to determine respiratory characteristics (e.g., normal, abnormal, etc.). Of course, a variety of other techniques may be used to determine respiratory characteristics.

According to signal sampling theory, to properly characterize a wave (or other periodic behavior), sampling should be sufficient to avoid frequency aliasing. In general, for equally spaced sampling, the sampling frequency must be greater than twice of the maximum frequency of the sampled behavior. Sometimes, the maximum frequency is referred to as the Nyquist frequency, which is the highest frequency that may be accurately sampled, and is one-half of the sampling frequency.

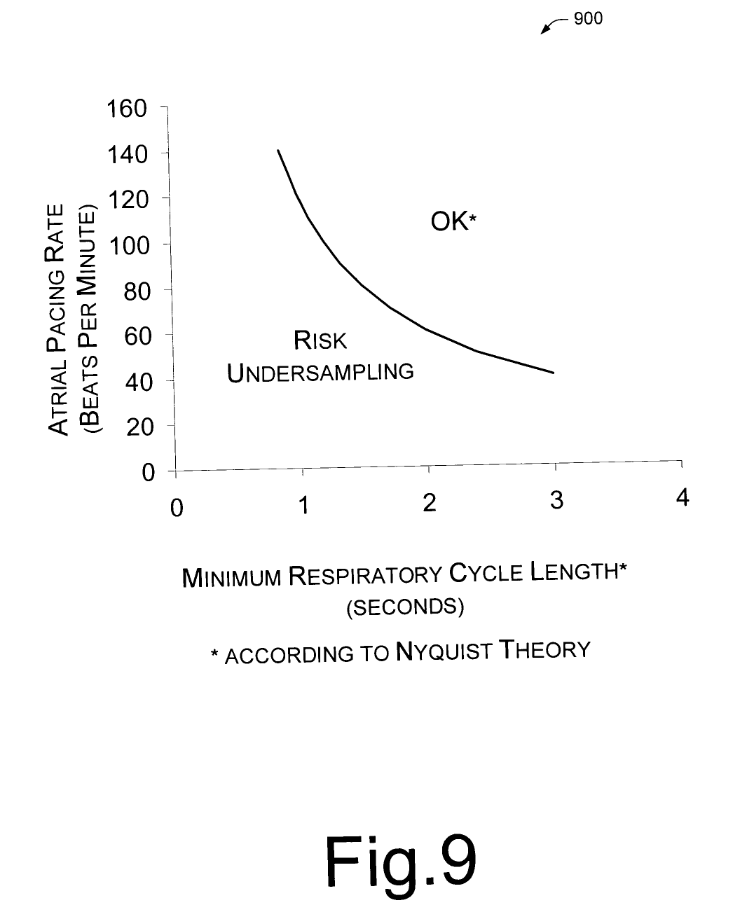

For the exemplary plot of 810, a sampling analysis indicates that approximately seven atrial paced stimuli (e.g., cardiac cycles) occur per respiratory cycle; hence the sampling frequency is well within the Nyquist criteria. For the exemplary plot of 820, such a series of AVI times may be representative of an inadequate sampling frequency. If such a doubt exists, a check may occur to determine the minimum respiratory cycle length that corresponds to the sampling frequency, which is often equal to the paced atrial rate. For example, FIG. 9 shows a plot 900 of atrial pacing rate in beats per minute versus minimum respiratory cycle length in seconds. The curve divides the plot into an under-sampling region and a region where sampling occurs at least according to Nyquist theory. Thus, if atrial pacing (e.g., sampling) occurs at approximately 120 beats per minute, then the respiratory cycle would have to be less than approximately 1 second to result in under-sampling. In this example, where atrial pacing occurs at approximately 120 beats per minute, the AVI times of the plot 820 either represent an abnormal respiration or respiratory cycle(s) that are less than approximately 1 second. Of course, in this example, patient history or other information may be used to determine that, in either instance, such respiration is abnormal. Loss of 1:1 AV conduction presents yet another alternative for irregular AVI times, wherein an atrial paced stimulus, resulting in atrial capture, may not produce a corresponding ventricular event (e.g., 2:1 AV block). Further, AV block can be more likely to occur when parasympathetic tone (e.g., vagal tone) predominates (e.g., which typically occurs during exhalation).

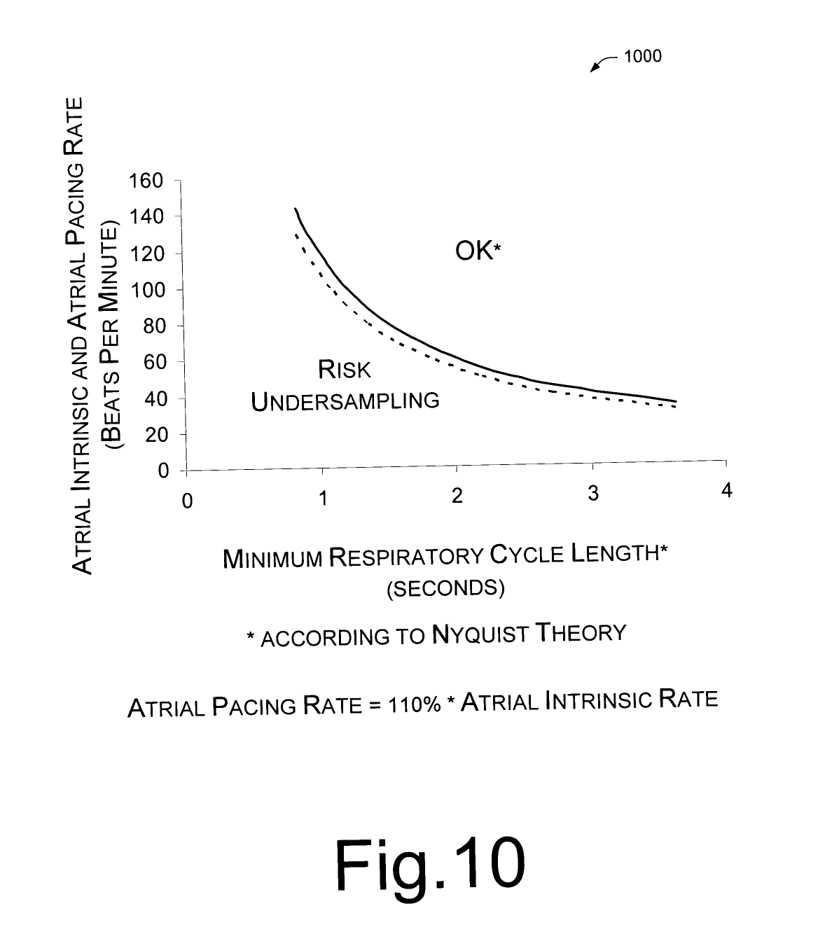

FIG. 10 shows a plot 1000 of atrial intrinsic rate and atrial pacing rate in beats per minute versus minimum respiratory cycle length in seconds. The dashed curve corresponds to atrial intrinsic rate while the solid curve corresponds to atrial pacing rate. In this example, the atrial pacing rate is set as a percentage (e.g., approximately 110%) of the atrial intrinsic rate. Of course, various other techniques may be suitable to set an atrial pacing rate based on an atrial intrinsic rate (e.g., an offset, etc.). The solid curve for atrial pacing rate divides the plot into an under-sampling region and a region where sampling occurs at least according to Nyquist theory. Such a plot, a representative equation, and/or a data table are optionally used to set an atrial pacing rate and to determine a minimum respiratory cycle length (or frequency). For example, the following equation may be used:
RCₘᵢₙ(s)=[0.5·(B·Rᵢ(Hz)+C)]⁻¹,
where Rₚ=B·Rᵢ+C, B and C are factors used to determine a paced atrial rate (Rₚ) based on an intrinsic atrial rate (Rᵢ) given in Hz, and the minimum respiratory cycle length (RCₘᵢₙ) is given in seconds.

Of course, some limits may exist for atrial paced rates. For example, if the atrial paced rate is too high, associated effects may confound efforts to discern a respiratory cycle or respiratory frequency on the basis of AVI times. Effects of atrial rate on AV conduction are known to exist. In general, AV conduction decreases as atrial rate increases and AV conduction increases as atrial rate decreases in a physiologic setting as associated with P waves. However, even in the presence of such effects, respiratory oscillations in AV conduction and/or AVI times may still be observed. For example, a study by Warner and Loeb, “Beat-by-beat modulation of AV conduction: I. Heart rate and respiratory influences”, Am. J. Physiol., 251: H 126-1133 (1986), which is incorporated by reference herein, indicates that, for a certain range of atrial paced rates in dogs, respiration induced oscillations in AVI times are observable. The Warner and Loeb study referred to this range as “low paced rates” (e.g., 125-245 beats per minute); whereas, at “intermediate paced rates” (e.g., 200-293 beats per minute), the Warner and Loeb study stated that respiratory-related variations in AV interval were reduced or absent. Thus, various exemplary methods described herein use atrial pacing at rates that are above an atrial intrinsic rate, that conform to sampling theory (e.g., Nyquist), and/or allow for observation of respiration induced oscillations in AVI times. Further, various exemplary devices described herein are capable of implementing such methods. With respect to typical rates for normal humans, a lower rate may be considered a rate less than or equal to approximately 45 bpm, an intermediate rate may be considered a rate greater than approximately 45 bpm to approximately 80 bpm, and a high rate may be considered a rate greater than approximately 80 bpm, wherein a rate of approximately 150 bpm is generally a rather realistic upper limit that is seldom reached in absence of vigorous exercise.

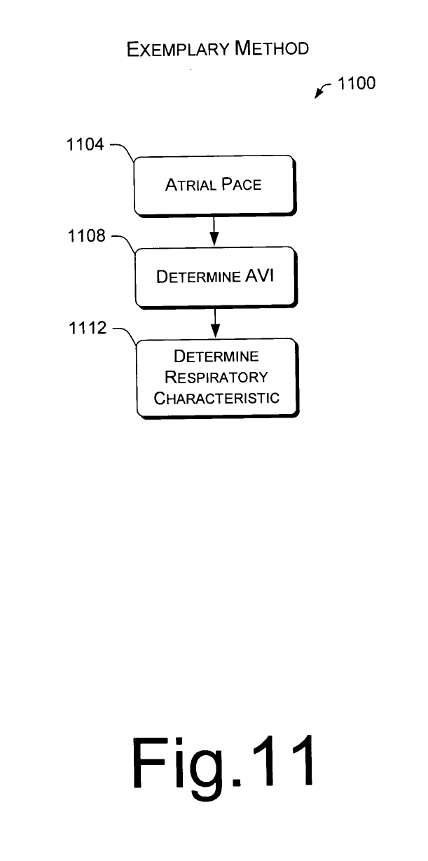

FIG. 11 shows a block diagram of an exemplary method 1100 for determining one or more respiratory characteristics as a function of one or more AVI times. In an atrial pace block 1104, atrial pace is delivered such that the atrial pace controls over any intrinsic atrial activity. Next, a determination block 1108 determines an AVI time, for example, based on the atrial pace and a corresponding sensed R wave. Another determination block 1112 follows that determines one or more respiratory characteristics as a function of the AVI time and optionally other AVI times or AVI criteria. For example, the exemplary method 1100 may compare the AVI time with one or more previously acquired AVI times and then determine that a patient is inhaling or exhaling. Of course, the atrial pace block 1104 and the determination block 1108 may form part of a loop to provide a series of AVI times which may be subsequently used in the determination block 1112 to determine one or more respiratory characteristics. Further, the atrial pacing rate may vary during iterations of the loop wherein the atrial pacing rate optionally remains above an atrial intrinsic rate, conforms to sampling theory (e.g., Nyquist), and/or allows for observation of respiration induced oscillations in AVI times.

In an alternative exemplary method an atrial pace block delivers an atrial pace such that the atrial pace controls over any intrinsic atrial activity. Next, a determination block determines an AVI time, for example, based on the atrial pace and a corresponding sensed R wave. Another determination block follows that determines one or more autonomic characteristics as a function of the AVI time and optionally other AVI times or AVI criteria. In this alternative, the autonomic characteristics optionally include autonomic balance or tone, a shift in autonomic balance or tone, a change in parasympathetic activity and/or a change in sympathetic activity. For example, the exemplary method may compare the AVI time with one or more previously acquired AVI times and then determine that a patient is experiencing a shift to parasympathetic or a shift to sympathetic. Of course, in this alternative, the atrial pace block and the determination block may form part of a loop to provide a series of AVI times which may be subsequently used in the determination block to determine one or more autonomic characteristics. Further, the atrial pacing rate may vary during iterations of the loop wherein the atrial pacing rate optionally remains above an atrial intrinsic rate, conforms to sampling theory (e.g., Nyquist), and/or allows for observation of autonomic changes via AVI times.

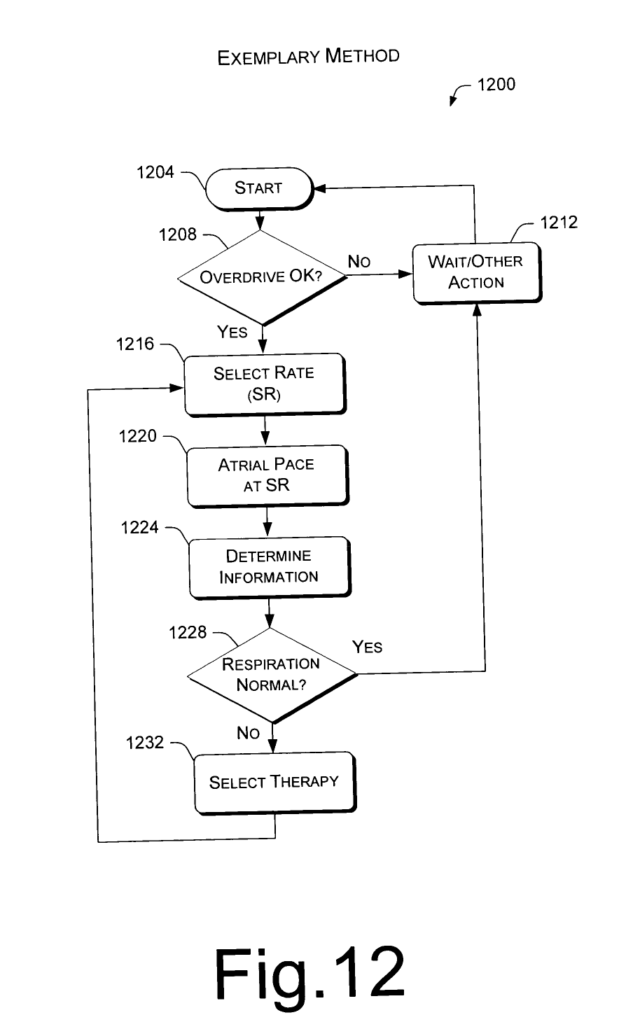

FIG. 12 shows a block diagram of an exemplary method 1200 that can use atrial pacing to determine whether respiration is normal. The method 1200 commences in a start block 1204. A decision block 1208 follows that decides whether atrial overdrive pacing may be implemented. A decision may be made as to sleep as well or may be inherent in the decision block 1208. For example, atrial pacing in sleep may be measured by an activity sensor or other circadian detection algorithm. If the decision block 1208 decides that atrial overdrive pacing cannot or should not be implemented, then the method 1200 continues in a wait or other action block 1212, wherein a return to the start block 1204 may occur. In the case that the decision block 1208 decides that atrial overdrive pacing may be implemented, then the method 1200 continues in a selection block 1216 that selects an atrial pacing rate. As discussed above, the selection of the atrial pacing rate may occur according to an equation, a data table, etc., that depends on the intrinsic atrial rate. Of course, selection may optionally occur on the basis of other factors. After rate selection, an atrial pace block 1220 begins pacing at the selected atrial pacing rate. A determination block 1224 follows that determines an AVI time, for example, based on the timing of an atrial pace and a corresponding sensed R wave. The atrial pace block 1220 and the determination block 1224 optionally form part of a loop to provide a series of AVI times. Another decision block 1228 follows that decides whether respiration is normal as a function of the AVI time and optionally other AVI times or AVI criteria. In the case that the decision block 1228 decides that respiration is normal, the exemplary method 1200 continues at the wait or other action block 1212. However, if the decision block 1228 decides that respiration is not normal (e.g., abnormal), then the method 1200 continues in a therapy selection block 1232. The therapy selection block 1232 optionally selects a therapy to treat the abnormal respiration. For example, if the abnormal respiration is characteristic of central apnea, then the selected therapy may involve an increase in atrial rate and/or delivery of another stimulus to the upper airway in case of obstructive sleep apnea or phrenic nerve stimulation in central sleep apnea. As shown, the exemplary method 1200 continues at the selection block 1216 where selection of any atrial pacing rate occurs.

Various exemplary devices and/or methods described herein aim to use AVI as a surrogate for respiration pattern. In particular, in many instances, the AVI is essentially decoupled from or independent of pacing rate such that a device can monitor respiration pattern regardless of pacing rate or intrinsic rate. While various examples refer to atrial pacing, various exemplary methods apply to instances where no pacing occurs and respiration pattern is inferred from, for example, PR interval. Further, through use of PR interval, various exemplary devices and/or methods may be implemented in absence of pacing or use of an implanted cardiac pacing device.

In instances where a chest impedance measurement is available, various exemplary devices and/or methods may more readily discriminate between central and obstructive sleep apnea. For example, AVI may be used to detect apnea and a chest impedance measurement (or other suitable measurement, e.g., pH, etc.) may be used to determine if the detected apnea is obstructive or central. During obstructive sleep apnea, significant chest movement typically exists whereas during central sleep apnea, little chest movement typically exists or is much less frequent when compared to obstructive sleep apnea. Even in the absence of another measurement, AVI or PR interval patterns may be analyzed for indications that distinguish central and obstructive sleep apnea.

In instances where use of PR interval is desired to determine respiratory pattern, use of a sampling frequency limit may determine whether an intrinsic atrial rate (see, e.g., the plot 900 of FIG. 9) will allow for an appropriate respiratory pattern determination. Similarly, such a limit may be used to determine whether a paced atrial rate will allow for an appropriate respiratory pattern determination.

### CONCLUSION

Although exemplary methods and/or devices have been described in language specific to structural features and/or methodological acts, it is to be understood that the subject matter defined in the appended claims is not necessarily limited to the specific features or acts described. Rather, the specific features and acts are disclosed as exemplary forms of implementing the claimed methods and/or devices.

1. A method comprising:
delivering one or more pacing pulses to an atrium;
determining one or more atrioventricular conduction interval times based on the one or more pacing pulses; and
determining a respiratory characteristic based at least in part on the one or more atrioventricular conduction interval times.

2. The method of claim 1, wherein the respiratory characteristic comprises a respiratory cycle length.

3. The method of claim 1, wherein the atrial pacing occurs at a frequency that is at least double a respiratory frequency.

4. The method of claim 1, wherein the atrial pacing occurs at a rate that varies with respect to an intrinsic rate.

5. The method of claim 1, wherein the atrial pacing occurs at a rate that varies with respect to time.

6. The method of claim 1, further comprising determining whether the respiratory characteristic indicates apnea.

7. The method of claim 6, further comprising discriminating between obstructive apnea and central apnea.

8. The method of claim 7, wherein the discriminating relies at least in part on a measurement related to chest movement.

9. The method of claim 7, wherein the discriminating relies at least in part on a known respiratory characteristic representative of obstructive apnea or central apnea.

10. A method comprising:
delivering one or more pacing pulses to an atrium;
determining one or more atrioventricular conduction interval times based on the one or more pacing pulses; and
determining an autonomic characteristic based at least in part on the one or more atrioventricular conduction interval times.

11. The method of claim 10, wherein the autonomic characteristic comprises an autonomic shift.

12. The method of claim 10, wherein the atrial pacing occurs at a frequency that is at least double an autonomic frequency.

13. The method of claim 10, wherein the atrial pacing occurs at a rate that varies with respect to an intrinsic rate.

14. The method of claim 10, wherein the atrial pacing occurs at a rate that varies with respect to time.

15. A method comprising:
determining one or more atrioventricular conduction interval times; and
determining a respiratory characteristic based at least in part on the one or more atrioventricular conduction interval times.

16. The method of claim 15, wherein the respiratory characteristic comprises a respiratory cycle length.

17. The method of claim 15, further comprising delivering one or more pacing pulses to an atrium, and wherein determining the atrioventricular conduction interval time comprises determining an interval from the pacing pulses to an intrinsic ventricular event.

18. The method of claim 15, further comprising determining whether the respiratory characteristic indicates apnea.

19. A method comprising:
determining one or more atrioventricular conduction interval times; and
determining an autonomic characteristic based at least in part on the one or more atrioventricular conduction interval times.

20. The method of claim 19, further comprising atrial pacing at a frequency that is at least double a respiratory frequency.

21. An apparatus comprising:
means for determining one or more atrioventricular conduction interval times; and
means for determining a respiratory characteristic based at least in part on the one or more atrioventricular conduction interval times.

22. An apparatus comprising:
means for determining one or more atrioventricular conduction interval times; and
means for determining an autonomic characteristic based at least in part on the one or more atrioventricular conduction interval times.

23. An implantable cardiac stimulation system comprising:
sensing circuitry operative to sense atrial and ventricular events;
a processor connected to the sensing circuitry and operative to determine one or more atrioventricular conduction interval times based on the atrial and ventricular events, wherein the processor is further operative to determine a respiratory characteristic based at least in part on the one or more atrioventricular conduction interval times.

24. The system of claim 23, wherein the processor is operative to determine whether the respiratory characteristic indicates apnea.

25. The system of claim 24, wherein the processor is operative to discriminate between obstructive apnea and central apnea.

26. The system of claim 23, further comprising a pulse generator operative to generate stimulation pulses for delivery to a patient's heart, and at least one electrode connected to the pulse generator and configured for implant within the patient to deliver the stimulation pulses to the patient's heart, wherein the processor is operative to determine atrioventricular conduction interval times based on the delivered stimulation pulses and corresponding sensed ventricular events.

27. An implantable cardiac stimulation system comprising:
sensing circuitry operative to sense atrial and ventricular events;
a processor connected to the sensing circuitry and operative to determine one or more atrioventricular conduction interval times based on the atrial and ventricular events, wherein the processor is further operative to determine an autonomic characteristic based at least in part on the one or more atrioventricular conduction interval times.

28. The system of claim 27, further comprising a pulse generator operative to generate stimulation pulses for delivery to a patient's heart, and at least one electrode connected to the pulse generator and configured for implant within the patient to deliver the stimulation pulses to the patient's heart, wherein the processor is operative to determine atrioventricular conduction interval times based on the delivered stimulation pulses and corresponding sensed ventricular events.
